(19) United States
(12) Patent Application Publication
(10) Pub. No.: US 2009/0035220 A1
(43) Pub. Date: Feb. 5, 2009

(54) HIGH THROUGHPUT SCREENING SYSTEM AND METHOD

(76) Inventor: Kevin Jones, Pomona, CA (US)

Correspondence Address: ITALIA IP, 3500 WEST OLIVE AVE., SUITE 300, BURBANK, CA, 91505, US

(21) Appl. No.: 11/832,658

(22) Filed: Aug. 2, 2007

(51) Int. Cl. A61K 49/00; A61P 43/00; C12M 1/00

(52) U.S. Cl. 424/9.2; 204/403.01

(57) ABSTRACT

A high throughput screening method for determining the pharmacological effect of a given agent, the method comprising, providing an assay plate with at least one sample well, where at least one well contains a volume of media and at least one zebrafish, introducing at least one agent to at least one of the wells with the media and zebrafish and incubating for a given amount of time, providing an electrical stimulus to the plate so as to promote a locomotor response in the zebrafish, and detecting the locomotor response of the zebrafish in response to the electrical stimulus in each well an agent was added to relative to a control well. Also disclosed is an assay plate that includes a first electrode located substantially at the center of the bottom portion of the well and a second electrode located substantially around the circumference of the well wall.

### BACKGROUND

This application relates generally to a high throughput screening (HTS) method. More specifically, this application relates to a HTS system and method which utilizes the locomotor response of zebrafish fry, Danio rerio, (and any other similar teleost) to an electrical stimulus as a means of measuring the pharmacological activity of a given agent.

### FIELD OF THE INVENTION

The rate of innovative pharmaceutical therapies that reach patients seems to be slowing: the number of new molecular entities submitted to the FDA has declined by about half since 1997. In a recent report, the FDA points to technological deficits in toxicology as one of the primary causes of this problem, noting that in many cases, the approaches of the last century are still being used to assess this century's drug candidates. New animal models are needed to test the safety of novel drug candidates, and the FDA estimated that 10% improvement in predicting failures before clinical trials would save about $100 million per drug in development costs. Ulrich, R. & Friend, SH Toxicogenomics and drug discovery: will technologies help us produce better drugs? Nature Rev. Drug Discov. 1, 84-88 (2002). In addition to outdated technologies, toxicology frequently suffers from being divorced from the drug discovery process—efforts to discover leads and improve their potency often occur independently from the assessment of toxicity.

To date most toxicological or more generally, pharmacological, assays performed with zebrafish are accomplished using fluorescent dye-based techniques in which a fluorescent dye is added to the fish water along with a small amount of test compound. Although this approach has been successful at identifying many toxic and or biological compounds, it is both cumbersome and narrow because it requires distinct assays to be developed for every organ system or cell type of interest. Moreover, current approaches are time consuming and thus do not easily lend themselves to the HTS approaches currently demanded by the pharmaceutical industry. Thus, there is a need for a technology that uses a whole animal model in a HTS to identify pharmacological activity of agents (also referred to herein as biological agents or compounds) at a very early stage in the drug discovery process.

### SUMMARY

This application discloses a method, system, and multi-well plate or assay plate to electrically stimulate zebrafish for the purpose of evoking a locomotor response. In this invention the locomotor responses evoked from zebrafish constitute a robust signal for HTS. Zebrafish respond to weak electrical stimuli with a brief locomotor response. Because the physical movement of zebrafish can be readily quantified, it is possible to integrate this locomotor response into a robust screening technology. The novel approach described herein is based on the finding that healthy zebrafish respond to electrical stimuli with a robust and consistent locomotor response, whereas animals whose health has been compromised by exposure to a toxic compound, will respond with smaller and/or shorter lasting responses. By quantifying these responses, it is possible to identify molecules that acutely and chronically impair the locomotor responses of zebrafish. This application discloses an HTS system and method that is suitable for rapidly identifying various forms of pharmacological activity exhibited by biological agents since the locomotor responses of the zebrafish are reflective of the overall health of the animal.

In particular, this application discloses a high throughput screening method for determining the pharmacological effect of a given agent, the method comprising: providing an assay plate with at least one sample well, where at least one well contains a volume of media and at least one zebrafish; introducing at least one agent to at least one of the wells with the media and zebrafish and incubating for a given amount of time; providing an electrical stimulus to the plate so as to promote a locomotor response in the zebrafish; and, detecting the locomotor response of the zebrafish in response to the electrical stimulus in each well an agent was added to relative to a control well.

In another embodiment, this application discloses a high throughput screening system for determining the pharmacological effect of a given agent by measuring the locomotor response of an organism to an electrical stimulus, the system comprising: an assay plate including at least one sample well and capable of receiving and administering an electrical stimulus to at least one sample well of said assay plate by connecting said assay plate to electrical stimulus generating and administering means through connection means; computing means for selecting the amplitude and duration of an electrical stimulus to apply to at least one sample well and wherein the computing means is connected to electrical stimulus generating and administering means through connection means; electrical stimulus generating and administering means responsive to said computing means for generating and administering an electrical stimulus to at least one sample well of an assay plate connected to said electrical stimulus generating and administering means through connection means.

Further embodiments of the system for determining the pharmacological effect of a given agent by measuring the locomotor response of an organism to an electrical stimulus include means for detecting the locomotor response of the organism following the administration of the electrical stimulus and means for analyzing the locomotor response of the organism following the detection of the locomotor response.

This application also discloses an assay plate, comprising at least one sample well having first and second electrodes placed therein, wherein said first electrode is substantially located at the center of the of the bottom portion of the well and wherein the second electrode is located substantially around the circumference of the well wall; ground means, and means for connecting the assay plate to electrical signal generating means.

### BRIEF DESCRIPTION OF THE DRAWINGS

The drawings, when considered in connection with the following description, are presented for the purpose of facilitating an understanding of the subject matter sought to be protected.

FIG. 1 is a schematic of a high throughput screening system described herein;

FIG. 2 is a top elevational view of an assay plate described herein;

FIG. 3 is a flowchart for describing a high throughput screening method described herein;

FIG. 4 is a graph illustrating the effect of stimulus duration on evoking a locomotor response in zebrafish;

FIG. 5 is a graph illustrating the effect of agents on locomotor response in zebrafish;

FIG. 6 is a graph illustrating the effect of stimulus frequency on evoking a locomotor response as demonstrated by the corresponding ammonia production in zebrafish; and,

FIG. 7 is a graph illustrating the effect of an agent on locomotor response as demonstrated by the corresponding ammonia production in zebrafish.

### DETAILED DESCRIPTION

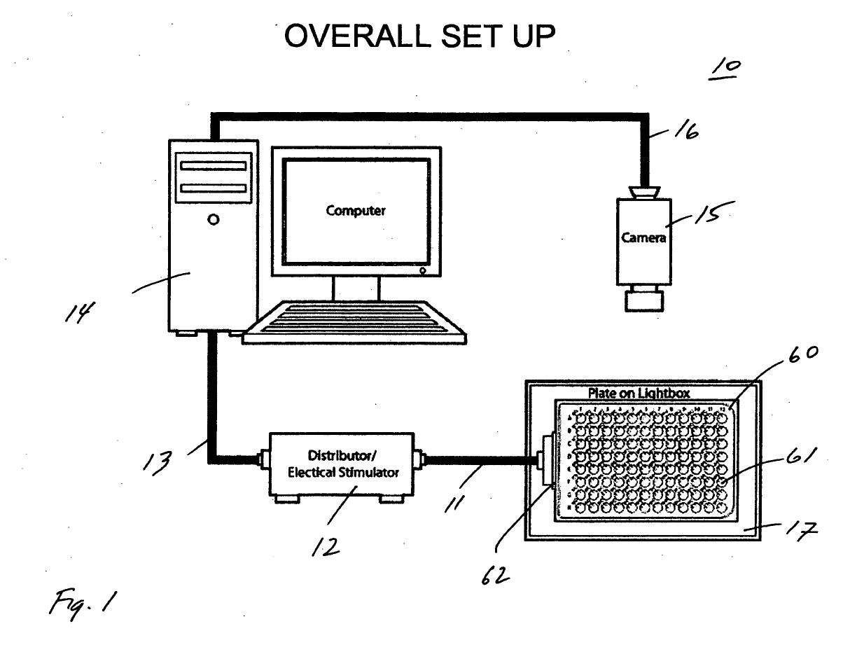

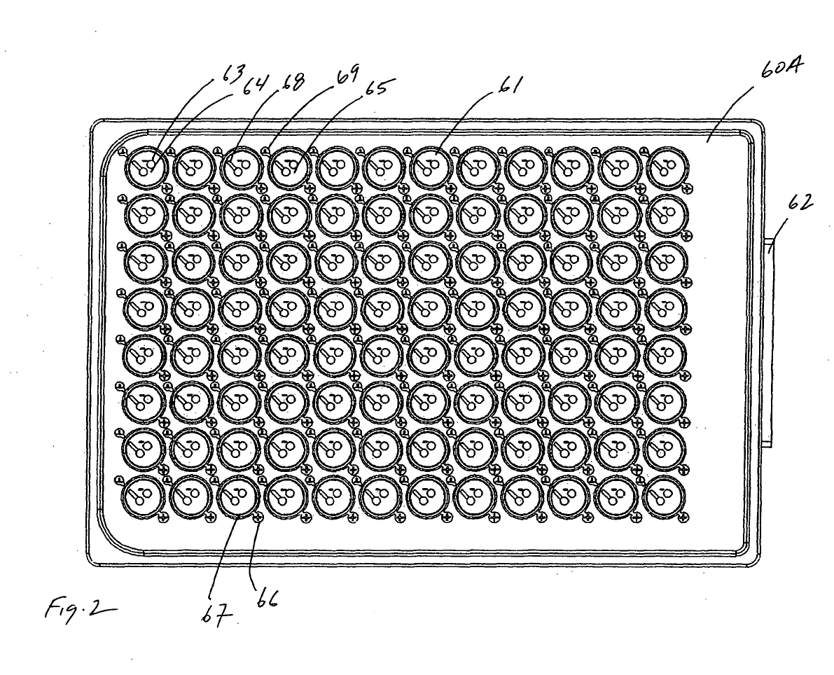

Referring to FIG. 1, shown therein and generally designated by the reference character 10 is a first embodiment of a high throughput screening system for determining the pharmacological effect of a given agent by measuring the locomotor response of a teleost to an electrical stimulus in accordance with the following description. The system 10 includes an assay plate 60 including at least one sample well 61 and or the standard 96-wells or any other number of wells that may be desired. The assay plate 60 is capable of receiving and administering an electrical stimulus through electrodes integrated with the wells 61 which allow for the electrical stimulation of the well as is common in the art. Preferably, the plate may include electrodes in the novel orientation as shown in FIG. 2. In FIG. 2, the assay plate 60A of the present disclosure is capable of receiving and administering an electrical stimulus through a connector 62 which may be of the type common in the art and may include a serial port, USB, ethernet, fire wire, or any other form of connection that allows the plate 60A to receive and transmit an electrical stimulus to at least one sample well 61, a row of wells, all the wells at one time, and any other combination desired. In the preferred embodiment, the assay plate 60A includes a first electrode 63 which is substantially located at the center 64 of the of the bottom 65 portion of each well 61. Preferably, the first electrode 63 is negatively charged. The plate 60A also includes a second electrode 66 which is located substantially around the circumference of the well wall 67 of each well 61. More specifically, the second electrode 66 is located at a position in the well wall 67 such that it is covered with a volume of media (about 100 to 400 ul in a standard 96-well plate) when used in the system and method described herein. Preferably the second electrode 66 is positively charged. The plate 60A also includes a ground electrode 68 for each well 61. Further, the plate 60A may include an additional test electrode 69 for monitoring the electrical stimulus delivered as a means of an internal check that the well was indeed stimulated at the proper settings. The electrodes can be made of materials common and in the art and include gold, platinum, palladium, chromium, molybdenum, iridium, tungsten, tantalum and titanium. The plate itself can be made of materials common in the art and include glass, quartz, cycloolefin, Aclar, polypropylene, polyethylene and polystyrene.

Continuing on with the HTS system 10 in FIG. 1, the assay plate 60 or 60A is connected by a first cable 11 to a distributor and electrical stimulator (as is common in the art) 12 that generates the electrical stimulus and administers the stimulus to the specified wells 61. The distributor and electrical stimulator 12 is connected to a computer 14 through connection means such as a second cable 13. The computer 14 allows the user to select various parameters of the electrical stimulus to be administered to the wells 61 including, the amplitude, duration, and frequency of the electrical stimulus, and the pattern and timing of the wells 61 to be stimulated. As also shown, the system 10 may also include a video camera 15 connected by a third cable 16 to the computer 14. The video camera 15 detects the locomotor response of the teleost following the administration of the electrical stimulus by using particle tracking technology available through vendors such as Noldus, Inc. This existing particle tracking technology permits the locomotor responses of up to 96 individual zebrafish (one per well) to be tracked and quantified simultaneously by including analysis software to measure the extent of locomotor response of each zebrafish. A lightbox 17 may also be used under the plate 60 or 60A and video camera 15 to ensure that the video camera 15 has sufficient light to detect the locomotor response. It should be appreciated that the certain actions of the various devices, be it the computer 14, the distributor and electrical stimulator 12, connector 62 of the plate 60 or 60A may be consolidated or separated in terms of function and still fall under the description setforth herein.

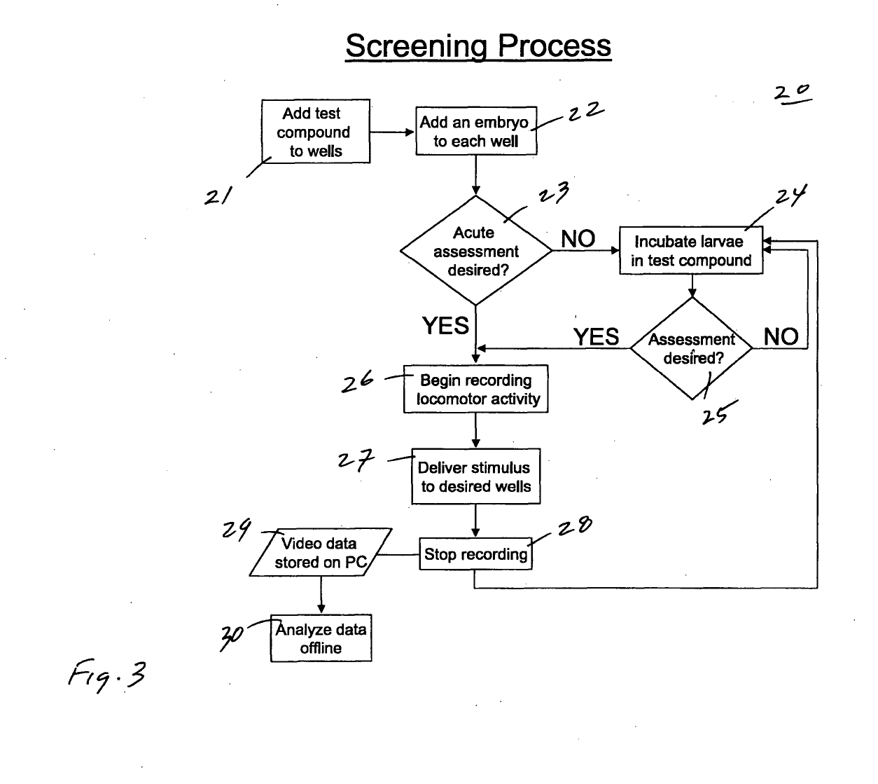

Referring now to FIG. 3, shown therein and generally designated by the reference character 20 is a first embodiment of a high throughput screening method for determining the pharmacological effect of a given agent by measuring the locomotor response of a teleost to an electrical stimulus in accordance with the following description. Specifics as to the conditions of the particular steps will be described more fully as part of the Examples provided below. As shown, the first step 21 is to add the test compound or agent to at least one of the wells 61 of the assay plate 60 or 60A containing embryonic media. Next 22, the teleost embryo is added to each well 61. A determination is made whether the desire is to test the agent for its acute effect 23 or its chronic effect. If the chronic effect is selected, the teleost larva is incubated 24 with the agent until assessment is desired 25. When the acute or chronic assessment is desired, the recording of the locomotor activity of the teleost is begun 26 by the video camera 15. The electrical stimulus is then delivered 27 to the desired wells 61 and the video camera 15 stops recording 28. At this stage, the data is stored on the computer 14 (PC) 29 and can be analyzed offline 30. Alternatively, the agent can be incubated 24 for an additional amount of time with the teleost to determine whether the agent has an effect on locomotor response over time. The subsequent steps as described above can then be followed until the true effect of the agent is determined to the satisfaction of the user.

### EXAMPLES

To create the graphs in the subsequent Examples, the initial conditions consisted of harvesting fertilized zebrafish eggs and placing them into embryo media and housed in an incubator with a constant temperature of 28.5° C. for the first several days of development, and throughout the course of experimentation. Embryo media consists of ultrapure water with 5 mM NaCl, 0.17 mM KCl, 0.33 mM CaCl₂, 0.3 mm MgSO₄ added. Embryo media was be used to make all final dilutions of test agents.

After the first 24 hours of incubation, the zebrafish larvae were treated with pronase solution (2 mg/ml in Embryo Medium) for several minutes to remove the larvae from their chorion. The larvae were then washed several times in embryo media to remove the pronase and a single larva was placed into one well of the 96-well plate containing a minimum volume of 150 ul of media or experimental solution (solution with the agent). The 96-well plates were maintained in incubators at 28.5° C. and were removed from incubators only for testing or to refresh media.

I. Determining the Stimulus Duration to Evoke a Locomotor Response.

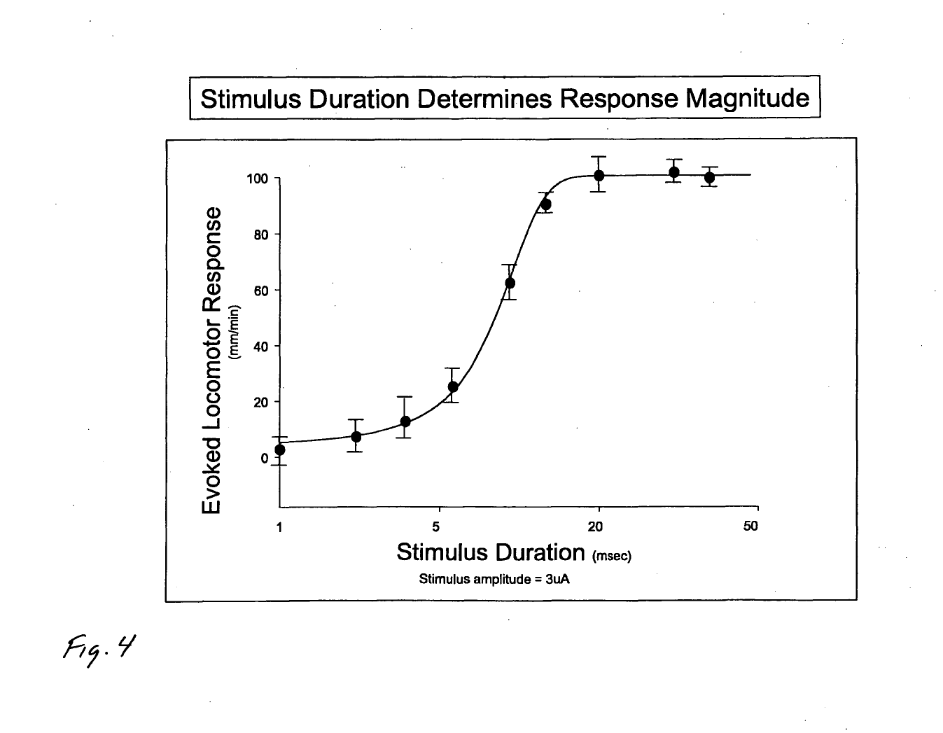

FIG. 4 is a graph illustrating the effect of stimulus duration on evoking a locomotor response in zebrafish. Utilizing the system 10 and general method 20 in FIGS. 1 and 2 wherein a video camera was used to detect the visual locomotor activity, the graph indicates that the locomotor response increased as the electrical stimulus duration increased until a maximum locomotor response was obtained at approximately 20 msec—where the stimulus amplitude maintained constant. Based on this data, conditions to evoke a maximum locomotor response were determined (20 msec at 3 uA) and applied in the following Example below.

II. Screening Compounds for Toxic Activity using Visual Detection of the Locomotor Response

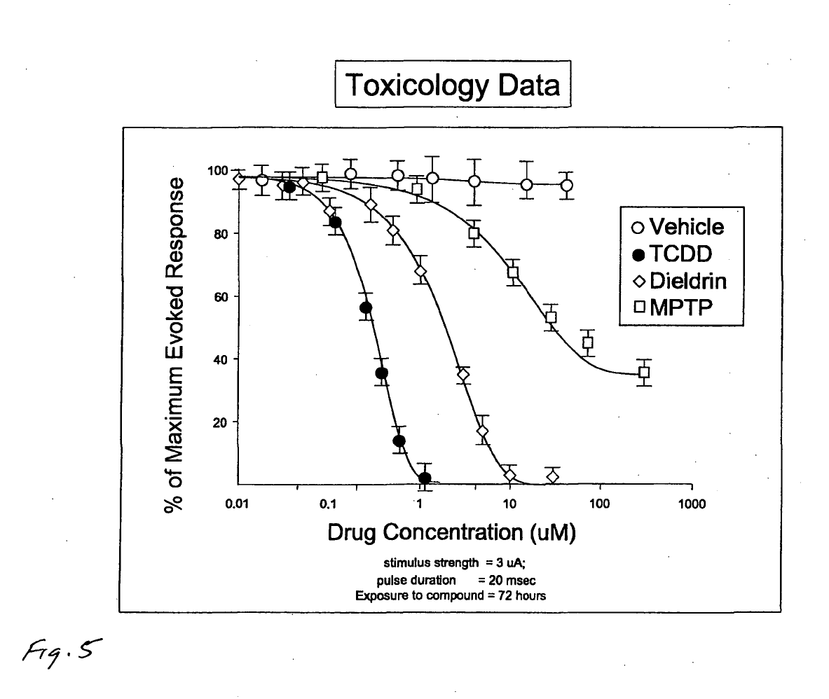

Referring now to FIG. 5, agents were added to the wells in the different concentrations to determine at what concentration locomotor response of the zebrafish was substantially inhibited. Again, the conditions of the stimulus were maintained at 20 msec at 3 uA and followed the system 10 and method 20 of FIGS. 1 and 2. In this example, the chronic effect of the agent was determined as the agent and zebrafish were incubated together at 28.5° C. for 72 hours before the data was obtained. As can be seen in the graph, curves indicating the toxicity of the agents can be readily obtained when compared to the control (vehicle) and relative to the other agents. To clarify, TCDD is one of the most potently toxic dioxins and is used as a reference for all other dioxins; Dieldrin is a chlorinated hydrocarbon originally produced by Bayer AG as an insecticide; and, MPTP is a chemical that is related to the opioid analgesic drugs and causes Parkinsonian side-effects.

III. Stimulus Frequency Correlates with Ammonia Excretion and Locomotor Activity

Excretion of ammonia is a necessary consequence of protein breakdown. When proteins are converted to carbohydrates to provide energy, the amino group is removed and must be dealt with. In animals, the amino group is quickly oxidized to form ammonia. Zebrafish larvae excrete water soluble ammonia into the media in a way that directly correlates to the locomotor activity of the zebrafish. Thus, the total amount of ammonia excreted by the larvae can be correlated to the locomotor activity (in response to the electrical stimulus) of the zebrafish without having to resort to the visual detection method and system described above. Alternatively, the ammonia excretion assay described herein can be utilized as an internal check if the visual detection method of the locomotor activity is employed, and vice versa. The concentration of ammonia excreted by the larvae was quantified by taking a sample of the media following the Examples and assaying it with a commercially available colorimetric assay kit (Ammonia Assay Kit #A1000, Sigma Biochemical)

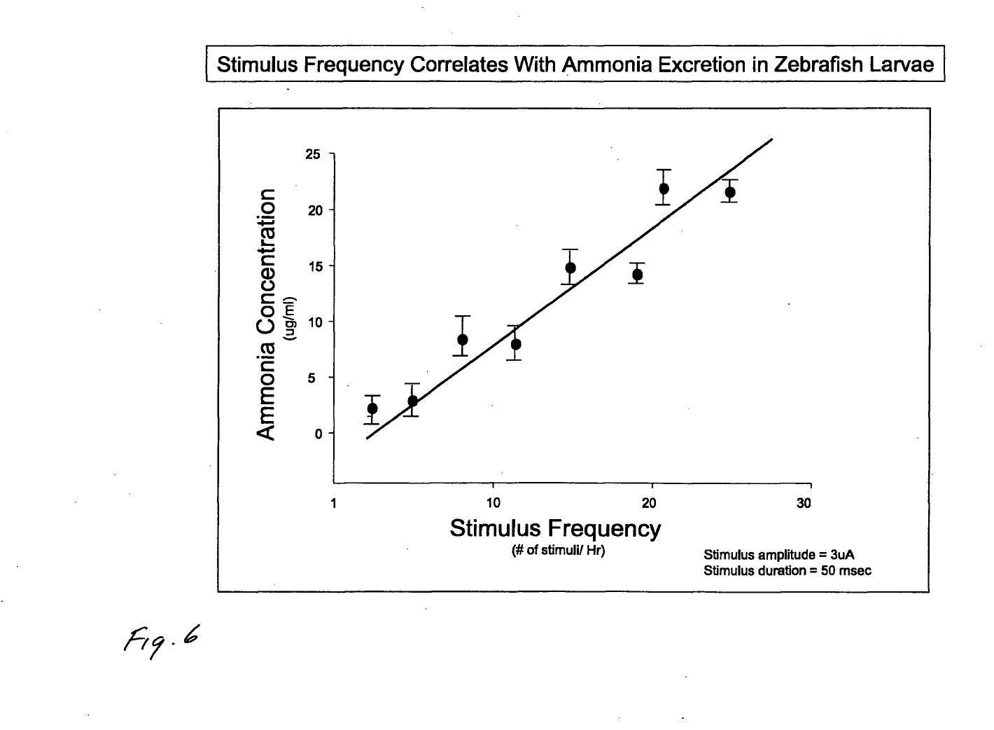

Referring to FIG. 6, and as described above, it can be seen that stimulus frequency was found to directly correlate to ammonia excretion and therefore locomotor activity in the zebrafish.

IV. Screening Compounds for Pharmacological Activity Using Ammonia Excretion as an Indicator of Locomotor Response

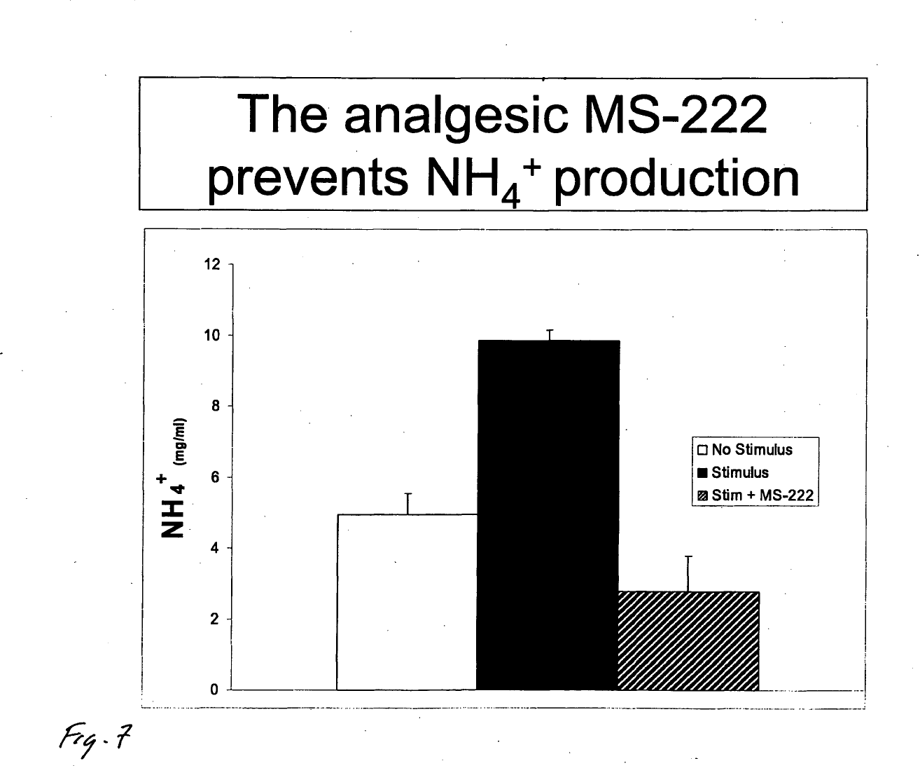

Referring now to FIG. 7, in this example 30 zebrafish larvae (4 dpf) were housed in each well of a 12-well plate. Larvae were exposed to media (n=8) or media containing a known anesthetic, MS-222 (Sigma Biochemical) (0.10 mg/mL) (n=4). Electrical stimului (3 uA, 50 msec) were applied to the groups of fish housed in the anesthetic and 4 of the groups of larvae housed in embryonic media. The last group was the control group and received no electrical stimulus (n=4). Stimuli were elicited at a frequency of 30 times per hour for 12 hours. After 12 hours, a 13 ul sample of the control media and the media with the anesthetic MS-222 was removed directly from the multi-well plate in which the zebrafish were housed. This sample volume was then assayed in the colorimetric assay (Ammonia Assay Kit #A1000, Sigma Biochemical) to determine the concentration of ammonia secreted by each animal. Results of this example are shown in the graph in FIG. 7 and are reported as the mean of 4 trials +/−standard error of the mean. The graph indicates that exposure to the anesthetic MS-222 decreases the locomotor response of zebrafish to an electrical stimulus based on the resulting decrease in the excretion of ammonia.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements. For example, based on the present disclosure, it should be readily understood that the Example of determining the stimulus duration to evoke a proper motor response could be utilized to determine mutant teleost. Such mutant teleost would be easily identified by their locomotor response falling outside the norm of a typical response at a given stimulus amplitude and duration. The mutant teleost could potentially have either an enhanced response to the stimulus or a decreased response to the stimulus. Nonetheless, the Example provided above would be a valuable tool in identifying such mutants. Likewise, in the second and third Examples involving detecting a change in the locomotor response in a teleost exposed to pharmacological agent as a means of determining the pharmacological activity of the agent, it should be readily understood that such assays would invariably identify mutant teleost that are resistant or hyperactive to various compounds. Such mutants would be identifiable by either an enhanced response to the compound or a decreased response to the compound when compared to the response of normal or wildtype teleost at a given concentration (where the stimulus amplitude, duration, and frequency is held constant). Finally, it should also be appreciated that the assay of measuring ammonia excretion in the third Example as an indicator of locomotor response to a given stimulus is directly related to and can be utilized to determine and measure the overall health of teleost. Healthy/normal teleost will have a reproducible level of ammonia excretion when reacting to a given electrical stimulus. This value can be used to compare compromised teleost against and identify if teleost are unhealthy.

What is claimed is:

1. A high throughput screening method for determining the pharmacological effect of a given agent, the method comprising:
providing an assay plate with at least one sample well, where at least one well contains a volume of media and at least one teleost;
introducing at least one agent to at least one of the wells with the media and teleost and incubating for a given amount of time;
providing an electrical stimulus to the plate so as to promote a locomotor response in the teleost; and,
detecting the locomotor response of the teleost in response to the electrical stimulus in each well an agent was added to relative to a control well.

2. The method of claim 1 wherein the plate includes at least two electrodes of opposite charge mounted within at least one well.

3. The method of claim 1 wherein the plate includes at least one sample well having first and second electrodes placed therein, wherein said first electrode is substantially located at the center of the of the bottom portion of the well and wherein the second electrode is located substantially around the circumference of the well wall;
ground means; and,
means for connecting the assay plate to electrical signal generating means.

4. The method of claim 1 wherein the method of detecting the locomotor response is visual.

5. The method of claim 4 wherein the visual locomotor response is detected by a video camera.

6. The method of claim 5 wherein the visual locomotor response detected by the video camera is quantified and compared to a control well.

7. The method of claim 4 wherein the video camera detects the locomotor response in each well of a multi-well plate at substantially the same time.

8. The method claim 1 wherein the method of detecting the locomotor response is through the administration of a labeling reagent to at least a portion of the media.

9. The method of claim 8 wherein the labeling reagent detects ammonia.

10. The method of claim 9 wherein the locomotor response detected by the ammonia labeling reagent is quantified and compared to a control well.

11. The method of claim 8 wherein the labeling reagent is a substrate for an enzymatic reaction.

12. An assay plate, comprising:
at least one sample well having first and second electrodes placed therein, wherein said first electrode is substantially located at the center of the of the bottom portion of the well and wherein the second electrode is located substantially around the circumference of the well wall;
ground means, and,
means for connecting the assay plate to electrical signal generating means.

13. The assay plate of claim 12 wherein the connecting means is a serial port.

14. The assay plate of claim 12 wherein the first electrode is negatively charged and the second electrode is positively charged.

15. The assay plate of claim 12 wherein the second electrode is located at a position in the well wall such that it is covered with a volume of media when used in an assay.

16. A high throughput screening system for determining the pharmacological effect of a given agent by measuring the locomotor response of a teleost to an electrical stimulus, the system comprising:
an assay plate including at least one sample well and capable of receiving and administering an electrical stimulus to at least one sample well of said assay plate by connecting said assay plate to electrical stimulus generating and administering means through connection means;
computing means for selecting the amplitude and duration of an electrical stimulus to apply to at least one sample well and wherein the computing means is connected to electrical stimulus generating and administering means through connection means;
electrical stimulus generating and administering means responsive to said computing means for generating and administering an electrical stimulus to at least one sample well of an assay plate connected to said electrical stimulus generating and administering means through connection means.

17. The system of claim 16, wherein the assay plate includes at least one sample well having first and second electrodes placed therein, wherein said first electrode is substantially located at the center of the of the bottom portion of the well and wherein the second electrode is located substantially around the circumference of the well wall;
ground means, and,
means for connecting the assay plate to said electrical stimulus generating and administering means.

18. The system of claim 16 wherein the system further comprises means for detecting the locomotor response of the teleost following the administration of the electrical stimulus.

19. The system of claim 18 wherein the means for detecting the locomotor response is a video camera.

20. The system of claim 18 wherein the system further comprises means for analyzing the locomotor response of the teleost following the detection of the locomotor response.
